(19) United States
(12) Patent Application Publication
(10) Pub. No.: US 2019/0321426 A1
(43) Pub. Date: Oct. 24, 2019

(54) COMBINATION THERAPIES WITH CANNABIS PLANT EXTRACT

(71) Applicant: TIKUN OLAM LTD, Tel Aviv (IL)

(72) Inventor: Ruth GALLILY, Jerusalem (IL)

(73) Assignee: TIKUN OLAM LTD, Tel Aviv (IL)

(21) Appl. No.: 16/346,901

(22) PCT Filed: Nov. 2, 2017
(86) PCT No.: PCT/IL2017/051197

§ 371 Date: May 2, 2019

Related U.S. Application Data
(60) Provisional application No. 62/416,325, filed on Nov. 2, 2016.

(51) Int. Cl. A61K 36/185; A61K 31/05; A61K 31/352; A61K 9/00; A61P 25/00

(52) U.S. Cl. CPC A61K 36/185; A61K 31/05; A61P 25/00; A61K 9/0014; A61K 31/352

(57) ABSTRACT

Provided are methods and formulations utilized in the methods which include cannabis plant extracts and copaxone

### TECHNOLOGICAL FIELD

The present disclosure relates to a combination therapy for treating autoimmune diseases and specifically for treating multiple sclerosis.

### BACKGROUND

Cannabinoids are chemical compounds that act on cannabinoid receptors. This group includes more than 100 different compounds, synthetic and those naturally occurring in plants (phytocannabinoids). Therapeutic effects of cannabinoids were previously described for a variety of indications. Medical cannabis research described many distinct kinds of cannabinoids, predominantly from Cannabis sativa and Cannabis indica plants, some in the form of extracts. Among numerous examples of cannabis extracts, certain cannabis extracts were described in US Patent Applications No. 2014/0259228 [1] and No. 2016/0073566 [2].

Glatiramer acetate (GA) also known as Copaxone (Teva pharmaceuticals) is used in reducing the frequency of relapses in patients with Relapsing-Remitting Multiple Sclerosis (RRMS), and in patients who have experienced a first clinical episode of Multiple Sclerosis (MS).

Compositions comprising cannabidiol (CBD) and GA and their use were described in WO2008120207 [3].

### PRIOR ART

### [1] US 2014/259228

### [2] US 2016/073566

### [3] WO2008/120207

[4] Gallily R et al., Overcoming the Bell Shaped Dose-Response of Cannobidiol by using Cannabis Extract enriched in Cannabidiol, Pharmacology & Pharmacy 2015; 6, 75-85.

### SUMMARY OF THE INVENTION

The present disclosure is generally based on the findings that a combination therapy comprising glatiramer acetate (GA) and at least one cannabis plant extract is effective in treating multiple sclerosis (MS), and is also effective in reducing side effects (or adverse events) associated with administration of GA, and specifically with side effects associated with subcutaneous (s.c.) injection of GA.

Glatiramer acetate (GA, Copaxone, copolymer 1) for injection is an approved drug for chronic or relapsing-remitting MS. The clinical and immunological effects of GA were extensively studied in experimental autoimmune encephalomyelitis (EAE), the experimental animal model for MS, and in human clinical studies. The commercial Copaxone is intended for subcutaneous use only (it is not administered intravenously) with a dosing schedule dependent on product strength. The most common adverse reactions to Copaxone, leading in at least 5% of patients to discontinuation of treatment, include: injection site reactions, urticaria, vasodilatation, rash, dyspnea, hypersensitivity and chest pain.

Specifically, the inventor of the technology has found that administration of a cannabis plant extract potentiates the effect of GA in treating MS and also reduces skin lesions associated with s.c. administration of GA (also referred to as local injection reaction).

While previous combinations of GA and CBD for nasal administration have been proposed for the treatment of MS (WO 2008/120207), they were found unsuitable for prolonged treatment of chronic MS due to irritation of nasal mucosa, and are considered more useful for immediate alleviation of an acute MS.

More specifically, as demonstrated herein, a combination therapy comprising GA and a cannabis plant extract, e.g., an extract comprising high levels of CBD, but not consisting solely of CBD, was effective in treatment of MS as compared to GA or a cannabis plant extract alone (see FIG. 3). In this instance, the GA has been introduced by s.c. administration and the cannabis extract—intraperitoneally (i.p.). The combination therapy was not only effective for the reduction of pathological manifestations of MS (see Example 1), but was further effective for the reduction side effects associated with GA s.c. injection (see Example 2).

Thus, in one of its aspects the invention provides a method for treating, preventing, ameliorating MS or delaying MS onset in a subject, the method comprising administering to the subject a combination therapy comprising a therapeutically effective amount of GA and a therapeutically effective amount of a cannabis plant extract.

It should be appreciated that the presently proposed combination therapies are applicable, in some embodiments, when GA is administered by injection, and s.c. injection in particular.

An important attribute of the presently conceived combination therapy is that it is more effective in terms of treating, preventing, ameliorating or delaying the onset of MS compared to each one of its components, GA or a cannabis extract, alone.

In yet another aspect, the present invention provides a method for reducing, inhibiting, attenuating or eliminating at least one side effect associated with administration of GA to a subject, said method comprising administering an effective amount of at least one cannabis plant extract to the subject, such that the effective amount is sufficient to reduce at least one said side effect. In the instances wherein the GA is administered by s.c. injection, said at least one side effect can be associated with s.c. injection of GA.

According to the present invention the subject to be treated with the presently proposed combination therapy is one which has been found or determined to possibly benefit from the treatment with GA, and for whom a therapeutic protocol has been tailored or proposed, specifying effective amounts of the GA and the at least one cannabis plant extract, such that the amount of one component is adjusted to or determined based on the amount of the other component or to any one other protocol parameter which may, inter alia, depend on subject health and personal factors as well as on time of administration, sequence and administration regimen.

More specifically, in certain cases the subject to be treated is one who, at the time of assessment, is treated with GA, and the amount and frequency of administration of the cannabis extract is determined in consideration of the concurrent GA treatment. In some embodiments, the subject is one who is predisposed, suspected or known to suffer from injectable GA-related side effects, whereby the administration of the cannabis extract, as defined herein, assists in reducing or diminishing such side effects.

Yet in other cases the at least one cannabis plant extract is administered prior to the administration of GA. In some embodiments, the at least one cannabis plant extract is administered immediately after administration of GA. In other embodiments, the two are administered simultaneously.

Unlike the GA component, the cannabis plant extract used in a combination therapy of the invention underlies a mixture of phyto-derived materials or compositions obtained from a cannabis plant, most often from Sativa, Indica, or Ruderalis species. It should be appreciated that the material composition and other properties of the extract may vary and further may be tailored to meet the desired properties of a combination therapy according to the invention.

As the cannabis plant extract is obtained by, e.g., extraction directly from a cannabis plant, it can include a combination of several naturally occurring compounds among them at least one natural cannabinoid, i.e., tetrahydrocannabinol (THC), cannabidiol (CBD), the two main naturally occurring cannabinoids, and further less abundant cannabinoids such as one or a combination of CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether) and others.

Of particular interest to the presently proposed combination therapies are cannabis plant extracts rich in CBD, or those with a low concentration of THC, which have been exemplified in form of Avidekel strain (see Examples 1 and 2).

As has been noted, the THC and CBD as referred to herein also denote isomers, derivatives, or precursors thereof, such as (−)-trans-Δ9-tetrahydrocannabinol (Δ9-THC), Δ8-THC, and Δ9-CBD, and also THC and CBD derived from their respective 2-carboxylic acids (2-COOH) (also THC-A and CBD-A, catalyzed by heat, light, or alkaline conditions).

As described herein, it was found that a combination therapy comprising an extract of a cannabis plant and GA has profound effects both therapeutically and in reducing side effects associated with injectable GA. It should be understood that reference to administration of GA with an extract of a cannabis plant in ‘combination’ or ‘together’ refers to a treatment schedule involving more than one type of therapy. In accordance with the present disclosure, combination therapy denotes a regimen involving administration of at least two substances in a single treatment cycle. As may be appreciated, the combination therapy requires an assessment of various parameters, inter alia, treatment schedule, predetermined ratio of the cannabis plant extract and GA (dosing), number of treatment cycles, and others. Assessment of the combination therapy may be carried out before onset of treatment. For example, the cannabis plant extract may be administered during the period at which a patient is treated with GA, at any time before administration of GA or at any time after administration of GA, provided that the later administration is at a time sufficient to yield an effective combination therapy.

The GA and the cannabis extract described herein can be administered and dosed by methods of the invention, in accordance with good medical practice, such as systemically, for example by parenteral, e.g. intravenous, intraperitoneal or intramuscular injection. In another example, the cannabis extract and GA can be introduced to a site by any suitable route including intravenous, subcutaneous, transcutaneous, topical, intramuscular, intraarticular, subconjunctival, or mucosal, and also oral, or intraocular administrations. The administration of the two components may be by the same or different modes of administration and at the same or different frequency, i.e. at the same or different time points.

The present disclosure also provides a pharmaceutical composition comprising GA and at least one cannabis plant extract, in many instances the at least one cannabis plant extract is rich in CBD and poor in THC, such as extracts from the strain Avidekel. In such compositions, wherein the GA and the cannabis extract are present in the same composition, the composition may be adapted for parenteral, oral, or transdermal administration. In many cases such compositions are adapted for s.c. injection, including a carrier adapted for s.c. injection. The above pharmaceutical compositions are applied for treating, preventing, ameliorating or delaying the onset of MS, in a subject suffering therefrom or who is predicted to suffer from said disease or who has been diagnosed to potentially develop the disease.

As described herein, the presently proposed combination therapy can be relevant to administration of injectable GA, which has been related side effects such as skin lesions or site reactions, or pain (Copaxone prescribing information by Teva Pharmaceuticals USA, Inc, North Wales, 2009).

The present invention further provides use of GA for the preparation of a composition to be administered in combination with at least one cannabis plant extract, or use of at least one cannabis plant extract for the preparation of a composition to be administered in combination with GA.

Still further, the invention provides use of at least one cannabis plant extract for the preparation of a composition to be administered in combination with GA.

According to the present invention, each of the components, the GA and the cannabis extract, act better in combination than alone, articulated herein by the terms ‘more effective’ or ‘potentiated’ effects. Such potentiated effects can have clinical manifestations of increased therapeutic effects or reduced side effects associated with each one of the components, and also in terms of therapeutically effective doses of the components when administered alone. When one of the components is said to potentiate the other, it is meant that that component is capable, when administered in combination, of increasing, strengthening, rendering stronger or of higher efficacy the effect of the other component administered in combination.

Thus, in yet another aspect, the invention provides an effective amount of injected GA, e.g., s.c. injected GA, for use in a method of administering to a subject an effective amount of at least one cannabis extract, wherein the effective amount of GA is selected to potentiate, increase, strengthen, render stronger or of higher efficacy at least one effect associated with the effective amount of the cannabis extract.

The invention further provides a kit comprising in separate reservoirs an effective amount of GA and an effective amount of at least one cannabis plant extract, the kit further comprising instructions for using said effective amount of GA and effective amount of at least one cannabis plant extract, in a combination therapy.

The invention further provides an effective amount of GA for use in a method of administering to a subject an effective amount of at least one cannabis plant extract, wherein the effective amount of GA is selected to potentiate, increase, strengthen, render stronger or of higher efficacy at least one effect associated with the effective amount of at least one cannabis plant extract.

The invention further provides an effective amount of at least one cannabis plant extract for use in a method of administering to a subject an effective amount of GA, wherein the effective amount of the at least one cannabis plant extract is selected to potentiate at least one effect associated with the effective amount of GA.

The invention further provides an effective amount of at least one cannabis plant extract for use in a method of administering to a subject an effective amount of GA, wherein the effective amount of the at least one cannabis plant extract is selected to reduce or diminish at least one side effect associated with the administration to a subject of an effective amount of GA.

The invention further provides use of a therapeutically effective amount of a cannabis plant extract obtained from a cannabis plant known as ‘Avidekel’ as described herein, in a method of treating MS.

The invention further provides an extract obtained from a cannabis plant known as ‘Avidekel’ as described herein, for use in the preparation of a composition for the treatment or prevention of MS.

Also provided are compositions or formulations comprising an extract of a cannabis plant known as ‘Avidekel’, as described herein, for the treatment or prevention of MS, wherein optionally said extract is the only active material used for said treatment or prevention. In some embodiments, the composition further comprises one or more additional drugs or actives known in the treatment or prevention of MS.

The present disclosure also provides a method for treating, preventing, ameliorating or delaying the onset of multiple sclerosis, in a subject suffering therefrom or who is predicted to suffer from said disease or who has been diagnosed to potentially develop the disease, said method comprises administrating to said subject a therapeutically effective amount of a cannabis plant extract obtained from a cannabis plant known as ‘Avidekel’ as described herein.

As noted herein, the present disclosure refers to MS (formerly known as disseminated sclerosis or encephalomyelitis disseminate), a chronic, inflammatory, demyelinating disease that affects the central nervous system (CNS). The disease onset usually occurs in young adults with a prevalence between 2 and 150 per 100,000 depending on specific population and country. MS is characterized by presence of multiple (at least two) neurological attacks affecting the CNS, manifested in the form of demyelinating lesions on brain magnetic resonance imaging (MRI). MS takes several forms, with new symptoms occurring either in discrete episodes (relapsing forms) or slowly accumulating over time (progressive forms). Most people are first diagnosed with relapsing-remitting MS (RRMS), and develop secondary-progressive MS (SPMS) after a number of years. Between episodes or attacks, symptoms can withdraw completely, although permanent neurological problems often persist, especially in advanced disease. RRMS occurs in about in 85% percent of the patients and SPMS in about 15%. It should be appreciated that the protocols, compositions and kits of the invention are applicable for the treatment of both MS forms, RRMS and SPMS, in children, young adults and adult subjects.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 shows EAE mice treated with an extract of a cannabis plant comprising 16% CBD and 1% THC (denoted as Avidekel) administered per os (p.o.).

FIG. 2 shows EAE mice treated with either an extract of Avidekel delivered intraperitoneally (i.p.) or Copaxone administered subcutaneously (s.c).

FIG. 3 shows EAE mice treated with an extract of Avidekel (i.p.) or Copaxone (s.c.) alone and in combination.

### DETAILED DESCRIPTION OF EMBODIMENTS

Before describing the invention it should be noted that it is not limited to herein described methods and experimental conditions, as well as the terminology used herein for describing particular embodiments is not intended to be limiting. Unless defined otherwise, all technical and scientific terms used herein have the meaning as commonly understood by one of ordinary skill in the art to which this invention pertains. Although any methods and materials similar or equivalent to those described herein can be used in the practice or testing of the invention, particular methods and materials are now described.

One main aspect of the invention is to provide a method for treating, preventing, ameliorating MS or delaying MS onset in a subject in need thereof, the method comprising administering to the subject a combination therapy comprising a therapeutically effective amount of GA and a therapeutically effective amount of a cannabis plant extract.

In some embodiments, the GA is administered by injection.

It further embodiments, the GA is administered by s.c. injection.

An important feature of the present combination therapy is that it is more effective for treating, preventing, ameliorating or delaying the onset of MS compared to each one of its components, GA or a cannabis extract, alone. The term ‘effective’ with respect to the presently proposed combination therapy applies to a number of situations: 1—when the GA and cannabis-based combination is more effective for a reduction primary and/or secondary symptoms of MS than each one of the components alone (at least one symptom); 2—when the combination is more effective for a reduction of GA adverse reactions (at least one symptom); and 3—when the combination is effective for a reduction of GA dose compared to GA being administered alone. The concept of being effective is further applied in the context of onset of a symptom, duration, severity, relapse and overall occurrence thereof.

Thus in certain aspects, the invention can be articulated as a method for treating, preventing, ameliorating or delaying the onset of MS in a subject in a need thereof, said method comprises administrating to said subject a combination therapy comprising a therapeutically effective amount of injected GA and a therapeutically effective amount of a cannabis extract, wherein the combination therapy is effective for treating, preventing, ameliorating or delaying the onset of the disease.

From another point of view, the invention can be also articulated as an effective amount of injected GA for use in a method of administering to a subject an effective amount of a cannabis extract, wherein the effective amount of GA is selected to potentiate at least one effect associated with the effective amount of a cannabis extract. In this context, the term ‘potentiate’ and ‘effective’ are analogous.

The present therapeutic concept using a combination therapy for MS, with its implied methods and compositions and kits, can be applied for treating a chronic MS or an acute MS, or for preventing development, deterioration or a relapse of MS.

In other words, the present therapies can be applied to a subject suffering from MS or a subject who is predicted to suffer from MS or who has been diagnosed to potentially develop the disease.

In this connection, a number of clinical tools have been developed for the prediction of MS predisposition, severity and relapse, such as examining vitamin D blood levels is used for the prediction of risk of developing MS, there are a number of immune markers for predicting severity and clinical course in MS, and more recently—genetic markers.

In some embodiments, the present combination therapy are applicable to the prevention of a relapse of MS, e.g., including reducing the frequency of relapses in patients with relapsing-remitting multiple sclerosis (RRMS).

More specifically, MS as a clinical entity is highly heterogeneous with symptoms varying from person to person and over the course of time. Classical MS includes:
Numbness or weakness in one or more limbs, typically unilateral, legs and trunk;
Partial or complete loss of vision, usually in one eye at a time, often with pain during eye movement;
Prolonged double vision;
Tingling or pain in various body parts;
Electric-shock sensations that occur with certain neck movements (Lhermitte sign);
Tremor, lack of coordination or unsteady gait;
Slurred speech;
Fatigue;
Dizziness; and/or
Problems with bowel and bladder function.

In some embodiments the combination therapy of the invention is effective for treating, preventing, ameliorating or delaying at least one symptom of MS.

As has been noted, most MS patients have a relapsing-remitting disease course (RRMS), certain patients with RRMS eventually develop a steady progression of symptoms, known as SPMS.

In some embodiments, the combination therapy according to the invention is effective for treating, preventing, ameliorating or delaying at least one symptom of MS relapse in RRMS, at least one symptom of SPMS.

In certain embodiments, the combination therapy is effective for treating, preventing, ameliorating or delaying problems with mobility and gait specifically associated. SPMS.

In some embodiments, the terms ‘preventing’ and ‘delaying’ are used herein to convey a reduction of risk of a recurrence of MS episode, specifically in populations at risk, including among others patients with family history of MS, certain viral infections (most notably Epstein-Barr virus), certain autoimmune diseases (e.g. thyroid disease, type diabetes and inflammatory bowel disease), smoking patients (smokers are more likely, to develop RRMS).

The terms ‘preventing’ and ‘delaying’ are further used to convey a reduction of secondary symptoms related to MS condition, including (among others):
Muscle stiffness or spasms;
Paralysis, typically in the legs;
Problems with bladder, bowel or sexual function;
Mental changes, such as forgetfulness or mood swings;
Depression; and/or
Epilepsy.

It is further conceived that the present invention provides a method for reducing, inhibiting, attenuating or eliminating at least one side effect associated with administration of GA to a subject, said method comprising administering an effective amount of at least one cannabis plant extract to the patient, such that the effective amount is sufficient to reduce at least one said side effect.

It is conceived that in numerous embodiments at least one side effect is associated with GA administered by injection. In some embodiments, the at least one side effect is associated with s.c. injection of GA.

Common side effects associated with GA (Copaxone) include:
injection site reactions (e.g., pain, redness, soreness, itching, swelling, or lump);
nausea, vomiting;
chills, weakness, fever or flu symptoms;
joint aches, body aches, including neck pain, back pain;
double vision;
headache;
increased urge to urinate;
swelling in hands or feet;
vaginal itching or discharge and/or
white patches or sores in the mouth or on your lips.

Immediate reactions can include:
flushing (warmth, redness, or tingly feeling);
chest pain;
fast heartbeat;
anxiety;
shortness of breath: and/or
itching.

Serious side effects include:
dizziness;
fainting;
infection (such as fever, persistent sore throat);
mental/mood changes (such as depression);
severe pain at the injection site;
shakiness (tremor); and/or
vision problems.

It is conceived that at least one of the above side effects (also adverse reactions) is reduced in terms of onset, occurrence or severity due to the added administration of an effective amount of a cannabis extract in concurrence with the administration of GA. The timing for administering each one of these agents are discussed hereinbelow.

It is further conceived that the subject to be treated with the combination therapy is one for whom a therapeutic protocol has been tailored, specifying effective amounts of the GA and the at least one cannabis plant extract, depending on subject health and personal factors as well as on time of administration, sequence and administration regimen. In other words, according to the invention the effective therapeutic doses of the cannabis and GA components in the combined therapy are subject to personalized dosing regimens determined by the treating physician.

Many MS patients are treated with GA in the form of Copaxone using regimen of 20 mg administered in a single s.c. administration. Thus in numerous embodiments daily doses of the components of the present combination therapy are: 20 mg GA in the form of Copaxone administered in a single s.c. administration, and at least one cannabis extract in the range of 1004050 mg in a formulation adapted for oral or dermal administration.

In further embodiments, the above regimens can comprise daily doses of 20 mg Copaxone administered in a single s.c. administration, and an extract of Avidekel in the range of 100-1050 mg in a formulation adapted for oral or dermal administration

Specifically, when Copaxone is administered in the form of 20 mg in a single s.c. administration, the cannabis extract (also Avidekel) is administered in a daily dose of 100, 200, 300, 400, 500, 600, 700, 800, 900, 1000, 1100, 1200, 1300, 1400, 1500 mg, or more, to achieve the therapeutic effects referred to above.

The cannabis extract the cannabis extract (also Avidekel, or more than one extract) can be administered before or after or simultaneously with the s.c. injection of Copaxone, in the form of oil, for example, for an oral or dermal (including s.c.) application.

MS patients treated with Copaxone also use 40 mg administered s.c. three times per week and at least 48 hours apart. Thus in further embodiments daily doses of the GA and the cannabis components in the combination therapy are: 40 mg GA in the form of Copaxone administered three times per week in at least 48 hours intervals, and at least one cannabis extract in the range of 100-1500 mg per day in a formulation adapted for oral or dermal, or s.c. administration. In numerous embodiments, such regimens can use a cannabis extract of Avidekel. In such cases Copaxone is administered s.c. in the form of 40 mg three times per week (i.e., 120 mg weekly dose), and at least one cannabis extract (e.g., Avidekel) as 100, 200, 300, 400, 500, 600, 700, 800, 900, 1000, 1100, 1200, 1300, 1400, 1500 mg per day (or 700, 1400, 2100, 2800, 3500, 4200, 4900, 5600, 6300, 7000, 7700, 8400, 9100, 9800, 10500 mg per week), or more, to achieve the therapeutic effects referred to above.

In such cases, the cannabis component can be administered together or apart, before or in between Copaxone injections.

Thus, in some embodiments, the subject to be treated is one who, at the time of assessment, is treated with GA, and the amount and frequency of administration of the cannabis extract is determined in consideration of the concurrent GA treatment. In some embodiments, the subject is one who is predisposed, suspected or known to suffer from injectable GA-related side effects, whereby the administration of the cannabis extract, as defined herein, assists in reducing or diminishing such side effects.

In some embodiments, the at least one cannabis plant extract is administered prior to the administration of GA. In some embodiments, the at least one cannabis plant extract is administered immediately after administration of GA. In other embodiments, the two are administered simultaneously.

In some embodiments, the at least one side effect is a skin lesion induced by s.c. administration of GA. As described herein, such skin lesion may be any lesion known in the art to result from local injection site reactions.

When referring herein to glatiramer acetate (GA, also known as Copolymer 1, Cop-1, the active ingredient of Copaxone—as marketed by Teva Pharmaceuticals) is meant a random polymer consisting of acetate salts of synthetic polypeptides, containing four naturally occurring amino acids: L-glutamic acid, L-alanine, L-tyrosine, and L-lysine, with the average molecular weight of 4.7-13 KDa. GA is identified by specific antibodies, its biological activity is determined by its ability to block the induction of EAE in mice.

Under Copaxone is meant the commercially available a clear, colorless to slightly yellow, sterile, nonpyrogenic solution for s.c. injection wherein each 1 mL contains 20 mg or 40 mg GA and 40 mg mannitol in pH of approximately 5.5 to 7.0.

In a broader sense, the term ‘GA’ (Co-1) denotes a synthetic analogue of myelin basic protein (MBP) believed to be important in the process of myelination of nerves in the nervous system, and therefore implicated in the pathogenesis of MS. Its therapeutic effects of in the treatment of MS are thought to be via immunomodulation and neuroprotection.

It is conceivable that GA analogues and derivatives acting by the same biological mechanism can be part of the same combination therapy. For example, Plovamer Acetate (also PA or Cop-2) is a structurally similar copolymer mixture of four amino acids of defined ratio, it has been rationally designed to have improved efficacy over GA. PA and GA share a similar mechanism of action, both competitively bind to MHC II and drive T-helper cell (Th)2-like responses. PA is undergoing Phase II testing for RRMS.

Thus, the term GA as used herein encompasses a variety of amino acid copolymers mimicking MBP with a common biological feature of alleviation of EAE.

The effect of oral administration of GA was tested in both rodents and primates in acute as well as in chronic/relapsing models of EAE. Oral GA was found to suppress acute EAE induced in rats. mice, and rhesus monkeys. Thus the term GA as used herein does not necessarily related to an injected GA, or s.c. injected GA, but is further applicable to GA administered via other routes, in particular the oral route.

In contrast, the cannabis plant extract used in the combination therapy of the invention underlies a mixture of a number of phyto-derived materials or compositions obtained from a dry resin-producing pistillate inflorescences of a female cannabis plant. The most advantageous types of cannabis in term of variety and content of cannabinoids belong to the species of Sativa, Indica, and Ruderalis,

Thus, in numerous embodiments at least one cannabis plant extract used in the combination therapy of the invention is an extract derived from flowers of a female C. sativa, C. indica, or C. ruderalis plant.

In further embodiments the cannabis plant extract is obtained from a cannabis plant known as ‘Avidekel’ (US Patent Application No. 2014/0259228 and US Plant Patent Application No. 2016/0073566).

As the material composition and other properties of the extract may vary and further may be tailored to meet the desired properties of a combination therapy according to the invention, the cannabis plant from which the extract is obtained may be selected from natural occurring cannabis plants and/or cultivars, including hybrid varieties of C. Sativa and C. indica for example. The term ‘cultivar’ generally refers to an assemblage of plants selected for desirable characteristics that are maintained during propagation, such as better survival, boosting of flavor, color and smell, or medicinal properties, with the aim of intensifying thereof.

It should be emphasized that the cannabinoid materials used in accordance with the present invention are not synthetic or semi-synthetic cannabinoids and are not delivered in a form of vesicles of phospholipid/ethanol/propylene glycerol system.

Further, in some embodiments, compositions of cannabinoid materials of the invention are based on at least one cannabis plant extract obtained directly from a cannabis plant. Such phyto-derived extracts typically include a combination of several naturally occurring compounds, among them at least one naturally occurring cannabinoid, and further nitrogenous compounds, amino acids, proteins, enzymes, glycoproteins, hydrocarbons, alcohols, aldehydes, ketones, fatty acids, esters and lactones, steroids, terpenes, non-cannabinoid phenols, flavonoids, vitamins and pigments, relative abundance of which differs between cannabis varieties.

The main natural cannabinoids are listed in Table 1 below.

TABLE 1
Main natural cannabinoids

| Type | Skeleton |
|---|---|
| Cannabigerol-type CBG | 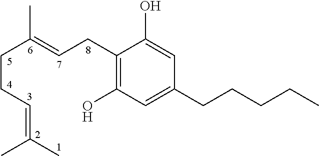 [SMILES: CCCCCc1cc(O)c(C/C=C(\C)CCC=C(C)C)c(O)c1] |
| Cannabichromene-type CBC | 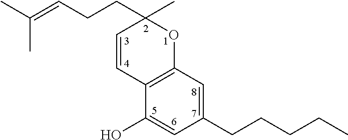 [SMILES: CCCCCc1cc(O)c2c(c1)OC(C)(CCC=C(C)C)C=C2] |
| Cannabidiol-type CBD | 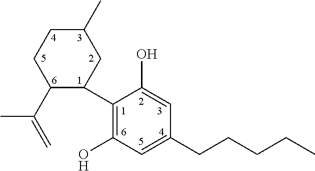 [SMILES: C=C(C)C1CCC(C)CC1c1c(O)cc(CCCCC)cc1O] |
| Tetrahydrocannabinol-and Cannabinol-type THC, CBN | 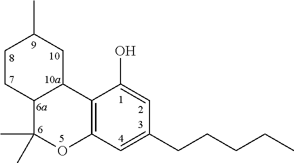 [SMILES: CCCCCc1cc(O)c2c(c1)OC(C)(C)C1CCC(C)CC21] |
| Cannabielsoin-type CBE | 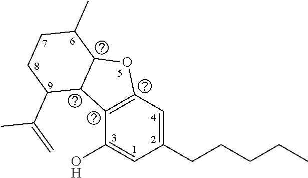 [SMILES: C=C(C)C1CCC(C)C2Oc3cc(CCCCC)cc(O)c3C12] |
| iso-Tetrahydrocannabinol-type iso-THC | 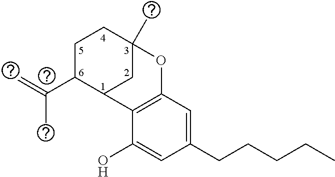 [SMILES: C=C(C)C1CCC2(C)CC1c1c(O)cc(CCCCC)cc1O2] |
| Cannabicyclol-type CBL | 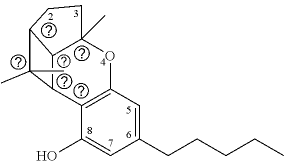 |
| Cannabicitran-type CBT | 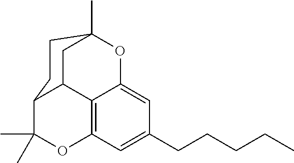 [SMILES: CCCCCc1cc2c3c(c1)OC(C)(C)C1CCC(C)(CC31)O2] |

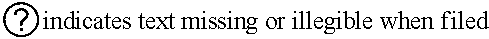

A single cannabis plant can comprise more than 80 types of specific cannabinoids form these classes.

Thus, in some embodiments, the at least one cannabis extract included in the combination therapy, due to its natural origin, can comprise a tetrahydrocannabinol-type and cannabinol-type (THC, CBN), a cannabidiol-type (CBD), a cannabigerol-type (CBG), a cannabichromene-type (CBC), a cannabielsoin-type (CBE), an iso-tetrahydrocannabinol-type (iso-THC), a cannabicyclol-type (CBL), a cannabicitran-type (CBT), a derivative, a precursor, or a combination thereof.

All classes derive from a cannabigerol-type compound differ mainly in how the precursor is cyclized. The classical cannabinoids are derived from their respective 2-carboxylic acids (2-COOH, also denoted with—A) by decarboxylation (catalyzed by heat, light, or alkaline conditions). Tetrahydrocannabinol and cannabidiol acid precursors, THC-A and CBD-A are also relevant to the present combination therapies.

A number of relevant phytocannabinoids are listed below:
THC (Tetrahydrocannabinol, including the two isoforms Δ9-THC, Δ8-THC and the acid form THC-A);
CBD (Cannabidiol and the acid form CBD-A);
CBN (Cannabinol);
CBG (Cannabigerol);
CBC (Cannabichromene);
CBL (Cannabicyclol);
CBV (Cannabivarin);
THCV (Tetrahydrocannabivarin);
CBDV (Cannabidivarin);
CBCV (Cannabichromevarin);
CBGV (Cannabigerovarin);
CBGM (Cannabigerol Monomethyl Ether).

The two main cannabinoids, in terms of relative content and psychotropic effects are THC and CBD. Apart from these two cannabinoids, the phyto-derived extracts included in the combination therapy are likely to comprise also THC and CBD metabolites, e.g., 11-Hydroxy Δ⁹-THC (active metabolite) and Δ⁹ Carboxy THC (inactive metabolite), and 7-COOH-CBD derivative, or 7-hydroxy CBD (7-OH-CBD), in various proportions.

In numerous embodiments the phyto-derived extracts included in the combination therapies can further comprise other cannabinoids, e.g., CBN, CBG, CBC, CBL, CBV, THCV, CBDV, CBCV, CBGV, and still further comprise precursors and acid forms, e.g., THCA, CBDA (also 2-carboxylic acids, 2-COOH), and other derivatives, e.g., CBGM, or any combination thereof.

Of potential relevance to the presently suggested combination therapies is another group of actives of plant origin, i.e., terpenes (also terpenoids). Terpenoids are flavor and fragrance components Generally Recognized as Safe by the US Food and Drug Administration and other regulatory agencies. They have been associated with unique therapeutic effects contributing to increased therapeutic index of cannabis-based extracts.

With respect to the two main cannabinoids, THC and CBD, it is conceived that in some embodiments the cannabis plant extract can be characterized as being rich in CBD. In some embodiments, the extract can comprise at least 10%, at least 11%, at least 13%, at least 14%, at least 15%, at least 16%, at least 17%, at least 18%, at least 20%, or at least 25% CBD (wt), or more.

In yet other embodiments, the cannabis plant extract can be characterized as having a low concentration of THC, e.g., being less than 5%, less than 4%, less than 3%, less than 2% or less than 1% (wt) THC.

In certain embodiments, the cannabis plant extract can be rich in CBD and poor in THC, for example comprising CBD in the range of 15-18% and THC in the range of 0.5-3.75% (w/w).

In yet other embodiments, the cannabis plant extract can comprise 16-18% CBD and 0.5-1% THC.

As has been noted, the THC and CBD as referred to herein also denote isomers, derivatives, or precursors thereof, such as (−)-trans-Δ9-tetrahydrocannabinol (Δ9-THC), Δ8-THC, and Δ9-CBD, and also THC and CBD derived from their respective 2-carboxylic acids (2-COOH), THC-A and CBD-A.

In some embodiments, the cannabis plant extract is an extract obtained from a cannabis plant described in US Patent Application No. 2014/0259228 and in US Plant Patent Application No. 2016/0073566. In some embodiments, the cannabis plant extract may be prepared by a method described in Gallily R et al., 2015 [4].

In some embodiments, the cannabis plant extract is obtained from a cannabis plant known as ‘Avidekel’ (US Patent Application No. 2014/0259228 and US Plant Patent Application No. 2016/0073566).

The term ‘extract’ (also referred to as ‘concentrate’) refers herein to a phyto-material obtained as a product of separation (or isolation) of one or more substances from a plant or a plant material using a solvent, or by using any other extraction technique known in the art. The extract may be obtained by extraction the plant material with water or a water containing solvent, or from a non-aqueous system comprising at least one suitable solvent, e.g., pentane, decane, cyclohexane, hexane, petroleum ether, chloromethane, ethanol, butanol, acetone, dichloromethane, chloroform, isopropanol, propanol, ethyl acetate, methanol, butylene glycol, propylene glycol, pentylene glycol, glycerol, ethers, oils, water or any other suitable solvent, and any mixture of these solvents. Extraction may be done by any conventional method in the art or such methods specifically suitable for extracting cannabis components. In accordance with some embodiments, the extraction is by supercritical CO₂ extraction, heating or ethanol extraction.

The plant may be subjected to extraction as a fresh plant (e.g. as a whole plant) or the plant may be a priori processed by any one of drying, semi-drying, cutting, chopping, grinding, powdering and the like.

The cannabis plant extract and GA described herein can be administered and dosed by the methods of the invention, in accordance with good medical practice, such as systemically, for example by parenteral, e.g. intravenous, intraperitoneal or intramuscular injection. In another example, the cannabis plant extract and GA can be introduced to a site by any suitable route including intravenous, subcutaneous, transcutaneous, topical, intramuscular, intraarticular, subconjunctival, or mucosal, e.g. oral, intranasal, or intraocular administration. The administration of the two components may be by the same or different modes of administration and at the same or different frequency, i.e. at the same or different time points.

In accordance with the present disclosure, administration of GA and the cannabis plant extract is at different dosage forms. In other words, GA and the cannabis plant extract are not present in the same composition. As detailed above, various modes of administration are known in the art, including, but are not limiting to, topical administration, enteral administration, parental administration. For example by injection (e.g., using a subcutaneous, intramuscular, intravenous, intraperitoneal, or intradermal injection), infusion (e.g. intraperitoneal), transdermal, transmucosal, intranasal administration, by inhalation or sublingually. For example, GA may be parenterally administered, by s.c. administration and the cannabis plant extract may be administered orally, sublingually, by injection (such as s.c.), by inhalation, by smoking intranasal transdermal or topical (topical administration being preferable at the site of injection).

In some embodiments, the GA and the cannabis plant extract are administered in different routes. In some embodiments, the GA is s.c. administrated. In some other embodiments, the cannabis plant extract is administrated topically, orally, sublingually or by inhalation. In some other embodiments, the cannabis plant extract is nasally administrated. In some further embodiments, the cannabis plant extract is administered by intraperitoneal administration. In some further embodiments, the cannabis plant extract is administered by transdermal administration. In some further embodiments, the cannabis plant extract is administrated by suppositories. As appreciated, the cannabis plant extract may be administered as drops, spray, or by nebulation. In some further embodiments, the cannabis plant extract is sprayed into the nose or mouth. In some embodiments, GA is s.c. administrated and the cannabis plant extract is nasally administrated. In some further embodiments, GA is s.c. administrated and the cannabis plant extract is administered by transdermal administration. In some further embodiments, GA is s.c. administrated and the cannabis plant extract is administrated by suppositories. In some embodiments, the cannabis plant extract is topically administrated, preferably at the site of injection. As described, GA may be injected s.c. into the abdomen, thigh (right and/or left), arm (right and/or left) and hip (right and/or left). In some embodiments, GA is administrated by s.c. injection into the abdomen and the cannabis plant extract is administrated by i.p. injection or infusion or by any other way which would increase the combined effect and reduce side effects associated with GA administration.

In some embodiments, each of GA and the cannabis plant extract are administered separately, i.e. not within the same composition/dosage form. Such an administration allows each component to be administered at a most effective site. In other words, GA may be administered s.c. and the cannabis plant extract may be administered topically or orally. In some embodiments, GA may be administered s.c. and the cannabis plant extract may be administered topically. In some further embodiments, the cannabis plant extract may be administered locally to the site of GA injection, preferably as a time window close to the injection (before or after).

In some embodiments, GA and the cannabis plant extract may be each administrated on a daily basis. In some other embodiments, the GA may be administered three time a week and the cannabis plant extract may be administrated on a daily basis. For example, GA may be administered at a daily dose of 20 mg by s.c. injection or at a dose of 40 mg injected three times a week and the extract either daily or also three times week on the same days the GA is administered.

In some other embodiments, the GA and the cannabis plant extract are administered simultaneously. In accordance with such embodiments, the GA and the cannabis plant extract are present in the same composition.

The present disclosure also provides a pharmaceutical composition comprising GA and at least one cannabis plant extract. In numerous embodiments, the at least one cannabis plant extract is rich in CBD and poor in THC.

In further embodiments the at least one cannabis plant extract comprises CBD in the range of 15-18% and THC in the range of 0.5-3.75% (w/w).

In further embodiments, the cannabis plant extract comprises 16-18% CBD and 0.5-1% THC.

In yet further embodiments the cannabis plant extract is obtained from a cannabis plant known as ‘Avidekel’ as described herein.

The pharmaceutical composition comprising GA and at least one cannabis plant extract is for use in treating, preventing, ameliorating or delaying the onset of multiple sclerosis, in a subject suffering therefrom or who is predicted to suffer from said disease or who has been diagnosed to potentially develop the disease.

In numerous embodiments the pharmaceutical composition can further comprise additional constitutes, excipients and/or drugs facilitating relevant to the symptoms, disease and administration route.

As detailed herein various modes of administration are known in the art to be applicable for administration of the composition comprising GA and the at least one cannabis plant extract. For example, topical administration, enteral administration, parental administration. More specifically, the composition comprising GA and the cannabis plant extract may be administered by injection (e.g., using a subcutaneous, intramuscular, intravenous, intraperitoneal, or intradermal injection), infusion (e.g. intraperitoneal), transdermal, transmucosal, orally, by suppository, by inhalation, or sublingually. In accordance with such embodiments, the composition comprising GA and the cannabis plant extract is not administered by intranasal administration.

In some embodiments, the composition comprising GA and the at least one cannabis plant extract is administered by an injection, e.g., by s.c. injection.

The invention can be further articulated in terms of a kit comprising in separate reservoirs an effective amount of GA and an effective amount of at least one cannabis plant extract, the kit further comprising instructions for using said effective amount of GA and effective amount of at least one cannabis plant extract in a combination therapy for the treatment of MS.

In some embodiments, the different reservoirs are different syringes or different formulation containers comprising the actives in solid or liquid or solution forms.

Another aspect of the invention is to provide use of GA for the preparation of a composition to be administered in combination with at least one cannabis plant extract.

Yet another aspect is to provide use of at least one cannabis plant extract for the preparation of a composition to be administered in combination with GA.

In some embodiments the at least one cannabis plant extract is rich in CBD and poor in THC, comprising CBD in the range of 15-18% and THC in the range of 0.5-3.75% (w/w).

In some embodiments, the cannabis plant extract comprises 16-18% CBD and 0.5-1% THC.

In some embodiments, the cannabis plant extract is obtained from a cannabis plant known as ‘Avidekel’ as described herein.

The cannabis plant extract may be obtained from a high CBD preferably low THC cannabis plant, most preferably from an Avidekel plant extract as presently exemplified.

In some embodiments, the composition of GA or the composition of the at least one cannabis plant extract is formulated separately from a composition of the at least one cannabis plant extract or composition comprising GA, respectively. In some further embodiments and as detailed herein, GA and the at least one cannabis plant extract are formulated within the same pharmaceutical composition.

As has been noted, in the present disclosure, treating, preventing, ameliorating or delaying MS refers to achieving a state of absence of disease activity in patients known to have the disease or who are predisposed to having the disease or who have been diagnosed as having the disease. In connection with MS, it is commonly used to refer to absence of active MS when this disease is expected to manifest again in the future. As MS is associated with symptoms occurring either in discrete episodes (relapsing forms) or slowly accumulating over time (progressive forms), a partial remission may be defined as 50 percent or greater reduction in the intensity and frequency of episodes or attacks. A complete remission may be defined as complete disappearance of all such manifestations of disease.

In some embodiments, treatment of MS refers to reducing the frequency of relapses in patients with relapsing-remitting multiple sclerosis (RRMS). In combination of the invention, each of the two components used in the combination therapy may potentiate the other. The term ‘potentiate’ as used herein refers to a pharmacologic response (effect) that is a result of the combination therapy and which is greater than each of the individual responses to each component or agent. When referring to potentiating of a therapeutic effect of GA by the cannabis plant extract, it should be noted to encompasses at least one of the following (i) an improvement of at least one diagnostic parameter in the patient treated with the combination therapy compared to a patient treated with GA alone or plant extract alone or (ii) obtaining the same improvement but with a lower GA dose or a longer doing interval.

The at least one diagnostic parameter may refer to any parameter used in diagnosis or prognosis of MS such as oligoclonal band. Oligoclonal bands (OCBs) are bands of immunoglobulins detected in a patient's blood serum, or cerebrospinal fluid (CSF). In MS, OCBs of immunoglobulin G antibodies are usually detected. In addition, at least one parameter may refer to any criteria known in the art for diagnosis or prognosis of MS such as Poser criteria or McDonald criteria. As appreciated, each one of these criteria refers a set of rules yielding conclusions related to the patient status. For example, the Poster criteria refers to a set of rules that can yield five conclusions: CDMS, LSDMS, CPMS, LSPMS or noMS, defined as follows: (1) CDMS—clinically definite MS: needs two attacks and some clinical or para-clinical evidences, (2) LSDMS—laboratory supported definite MS, showing oligoclonal bands and clinical or paraclinical evidences, (3) CPMS—clinically probable MS, with less restrict combinations, (4) LSPMS—Laboratory supported probable MS: only two attacks are enough to enter this category, and (5) no MS—there is no clinical evidence of having MS. The McDonald criteria maintained a scheme for diagnosing MS based on clinical grounds but also proposed that when clinical evidence is lacking, magnetic resonance imaging (MRI) findings can be used and this criteria may be used to facilitate the diagnosis of MS in patients who present with their first demyelinating attack and significantly increase the sensitivity for diagnosing MS without compromising the specificity.

The terms ‘treatment or prevention’ refers to the complete range of therapeutically positive effects of administrating to a subject including inhibition, reduction of, alleviation of, and relief from, a condition as detailed herein. More specifically, treatment or prevention of relapse or recurrence of the disease includes the prevention or postponement of development of the disease, prevention or postponement of development of symptoms and/or a reduction in the severity of such symptoms that will or are expected to develop. These further include ameliorating existing symptoms, preventing—additional symptoms and ameliorating or preventing the underlying metabolic causes of symptoms.

It should be appreciated that the terms ‘inhibition’, ‘reduction’, or ‘attenuation’ as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

As described herein, administration of injectable GA may be associated with side effects such as skin lesions or site reactions, or pain, such as a permanent indentation under the skin (lipoatrophy or, rarely, necrosis) at the injection site may occur, due to local destruction of fat tissue. This is also indicated in Copaxone prescribing information by the recommendation to follow proper injection technique and monitor any skin changes. The presently proposed combination therapy was surprisingly found to be associated with a reduction of these skin lesions were observed.

A ‘skin lesion’ or ‘injection site reaction’ as described herein refers to local skin reaction that occur upon administration of GA. Such lesion or reaction occurs usually when the drug escapes into the skin, resulting in an abnormal growth or different appearance compared to the skin around it. Exemplary skin lesion or site reaction include but not limited to a scar, edema, pain, redness, soreness, itching, swelling, or hard lump. In addition and in accordance with the present disclosure, skin lesion or site reaction also refer to an open wound that does not heal or appearance of blood clot. As such, the combination therapy is particularly suitable for administration to patients who are suffering from a skin lesion following treatment with GA. In the context of the present disclosure, a reduction in a skin lesion refers to at least one of a reduction in the severity of a skin lesion, reduction in the frequency of appearance of a skin lesion, reduction in the number of skin lesions or prevention of a skin lesion.

The cannabis plane extract and GA may each be administrated to a subject in need thereof in an effective amount. As known, the ‘effective amount’ for purposes herein may be determined by such considerations as known in the art. The amount must be effective to achieve the desired therapeutic effect, depending, inter alia, on the type and severity of the disease to be treated and the treatment regime. As generally known, the effective amount depends on a variety of factors including for example the distribution profile within the body, a variety of pharmacological parameters such as half-life in the body, on undesired side effects, if any, on factors such as age and gender, and others. In the context of the present disclosure and as described herein, the effective amount of the cannabis plant extract and the effective amount of GA are selected such that the combination has the desired therapeutic effect.

### NON-LIMITING EXAMPLES

### Example 1: Suppression of EAE in SJL Mice by Cannabis Extract with or without GA

### Materials

Cannabis dry flower comprising 16% CBD, 1% THC (known as Avidekel) was obtained from Tikun Olam, Israel.
Proteolipid protein, (PLP) (139-151) were purchased from (GP, China)
CFA and Pertusis toxin (PT) were purchased from Sigma.

### Methods

SJL/J female mice were purchased from Harlen. Mice at the age of 6-7 weeks old were used to the in the experiments described below. All the experiments were done in accordance with the protocol approved by the Ethics Committee of the Hebrew University of Jerusalem. The mice were housed in cages with free access to food and water. They were maintained in 12 hr. light/dark cycle at room temperature.

Mice were immunized with PLP (139-151) emulsified in CFA together with pertussis vaccine according to the method specified in Hooke Laboratories protocols (http://hookelabs.com/protocols/eaeAISJL). PLP is used to induce relapsing-remitting (RR)-EAE model.

In the EAE experiments, SJL/j female mice at an age of about 6-7 weeks old were used. Mice were observed daily for the appearance of neurological paralytic symptoms (7 mice/group), and were scored in scale from 0-5 denoting as follows:

0 no neurological sign

0.5 distal limb

1 limp fail

2 loose of righting reflex (difficulty to turnover when laid in the back)

3 ataxin hind limp paralysis (hind limbs are dragged)

4. paralysis of the hind legs

5. full paralysis (immobility)

6. death of the mice

Following s.c. injection of PLP (139-151) mixed with CFA as well as two pertussis-toxin injections and appearance of paralysis signs in the mice (usually after 9-11 days after initiation of EAE) treatment of mice began.

The treatments were given daily, five times a week and usually continued for about 60 days. GA were given s.c (at an injection dose of 1 mg/mouse), and cannabis extracts were administrated i.p. (intraperitoneal).

Administration of PLP 139-151 induced 3 phases of paralysis and in one experiment it induced only one phase. As known, 3 peaks are considered more similar to physiological manifestations in humans, however, one peak is considered acceptable and is used in many MS studies.

### Statistical Evaluation

Raw p-value were obtained from exact one-tail Mann Whitney tests and adjusted for multiple comparisons (within each experiment) using Holm modification of the Bonferroni correction

### Results

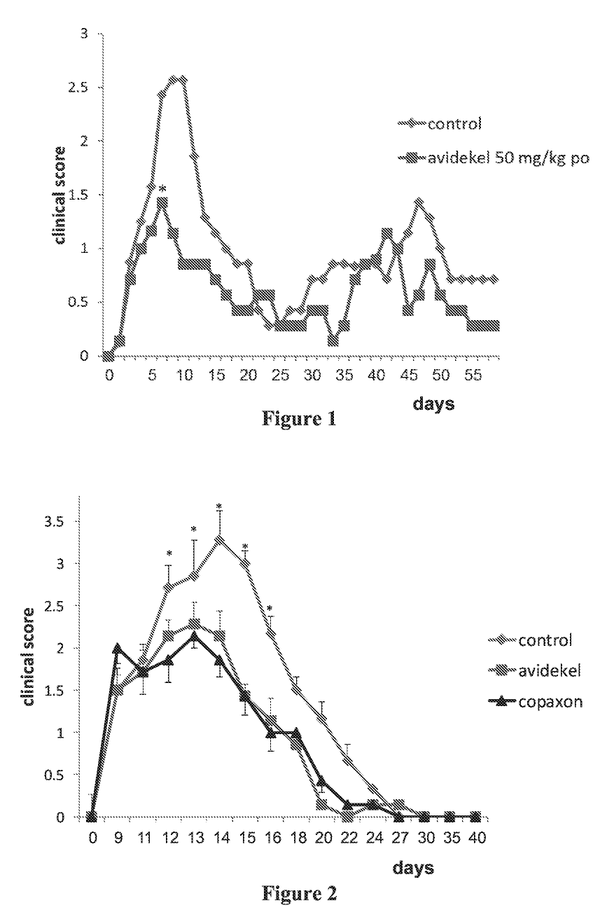

The effect of cannabis monotherapy is shown in FIG. 1, whereby the treatment with Avidekel plant extract (16% CBD, 1% THC) at 50 mg/Kg administered per os significantly reduced paralysis in the first peak by 45% and in the third peak by 46%.

Comparative effects of cannabis or GA as monotherapies are shown in FIG. 2. Specifically, only one peak of paralysis was observed after PLP injection. In all animals treated with GA (exemplified by Copaxone) or a cannabis plant extract (exemplified by Avidekel) clinical score were reduced by at least 32-35%.

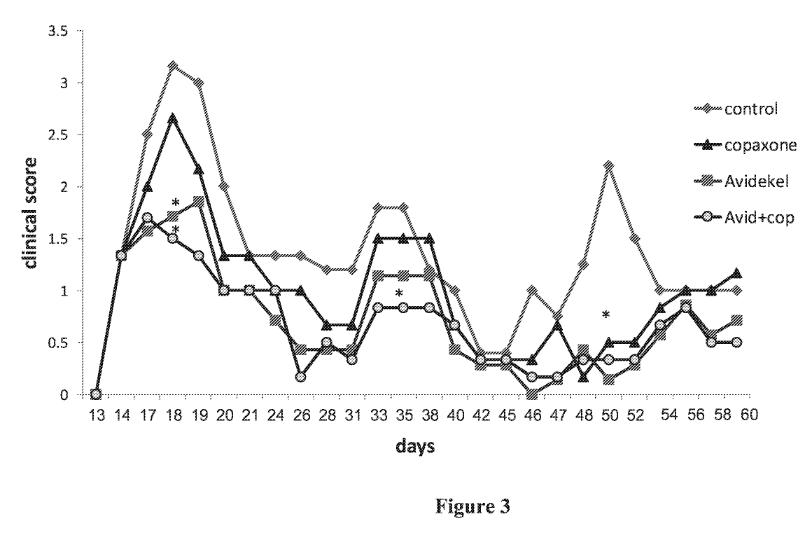

The effect of the combination therapy including a cannabis extract and GA is shown in FIG. 3. Mice were treated with Avidekel plant extract (16% CBD, 1% THC) administered i.p. or with GA as Copaxone administrated s.c., or with the combination of Avidekel and Copaxone administered as above FIG. 3 demonstrates significantly reduced clinical scores in mice treated with the cannabis plant extract alone or in combination with GA. The combination of the cannabis plant extract with GA had a more profound effect compared with each one of the components alone.

Specifically the results demonstrate that the cannabis extract from the plant denoted as Avidekel (enriched with CBD) significantly ameliorates the paralysis symptoms in EAE mice model.

### Example 2: Pathological Studies in SJL Mice by Cannabis Extract with or without GA

Studies of monotherapies (cannabis extract or Copaxone) and the combination therapy (cannabis extract plus Copaxone) also included histological studies of infiltration of immune cells into the spinal cord in EAE mice (i.e., MS core pathology). Sixty days following injection of PLP into control non-treated SJL female mice, a massive infiltration of immune cells into the white section of the spinal cord was observed in eosin/hematoxylin staining sections. In contrast, an almost total reduction of such infiltration into the spinal cord was observed following treatments with either Avidekel (the cannabis extract enriched with CBD), GA or GA plus Avidekel, showing effect at the tissue level of Avidekel alone and Avidekel with GA.

### Cutaneous Wound

Specific effects of the combination therapy (Avidekel plus Copaxone) were observed at the site of subcutaneous GA injection. In mice treated with the Copaxone only (2 out of 7 mice), a skin lesion developed on the mice back at the site of repeated Copaxone injections. No such lesions were observed in Avidekel-treated mice with or without Copaxone injection. The lesion (dermal wound) was characterized by skin swelling, open wound that did not heal and the appearance of blood clot.

These results demonstrated that co administration of Copaxone with the Avidekel extract prevented the formation of cutaneous wound characteristic of Copaxone when s.c. administered alone.

1.-51. (canceled)

52. A method for reducing, inhibiting, attenuating or eliminating at least one side effect associated with injecting glatiramer acetate (GA) to a subject suffering from an injection site reaction caused by the administration of GA,
said method comprises oral, transdermal or topical administering to the subject an effective amount of a composition comprising an extract of at least one cannabis plant simultaneously with, prior to or after injecting GA.

53. A method for reducing, inhibiting, attenuating or eliminating at least one effect of an injection site reaction associated with injecting glatiramer acetate (GA) in a subject suffering from an injection site reaction caused by the administration of GA,
said method comprises administering to the subject simultaneously with, prior to or after injecting GA an effective amount of an oral, topical or transdermal composition comprising an extract of at least one cannabis plant.

54. The method according to claim 52, wherein the subject is injected with GA for treating, preventing, ameliorating or delaying the onset of multiple sclerosis (MS).

55. The method according to claim 52, wherein said effect of the injection site reaction is selected from a scar, an edema, pain, redness, soreness, itching, swelling, and a hard lump.

56. The method according to claim 52, wherein said simultaneous administering of the composition of the cannabis plant extract is before or after injecting GA.

57. The method according to claim 52, wherein said administering of the composition of the cannabis plant extract or the composition per se involve topical or transdermal administering of the composition at the GA injection site.

58. The method according to claim 52, wherein the composition of the cannabis plant extract comprises 0.5-3.75% (w/w) THC and 14-25% CBD.

59. The method according to claim 52, wherein the at least one cannabis plant comprised in the extract is the cannabis strain designated herein as “Avidekel”.

60. A method for treating, preventing, ameliorating or delaying the onset of multiple sclerosis (MS) in a subject, said method comprising administering to the subject a combination therapy comprising co-administering of a therapeutically effective amount of glatiramer acetate (GA) and a therapeutically effective amount of an oral, topical or transdermal composition comprising an extract of at least one cannabis plant.

61. The method according to claim 60, wherein said treating, preventing, ameliorating MS further comprises reducing the frequency of relapses in the relapsing-remitting multiple sclerosis (RRMS).

62. The method according to claim 60, wherein said co-administering of the GA and the composition of the cannabis plant extract is simultaneous or sequential, the composition being administered prior to or after the GA.

63. The method according to claim 62, wherein the composition of the cannabis plant extract is topical or transdermal, and is co-administered at the GA injection site.

64. The method according to claim 60, wherein the composition of the cannabis plant extract comprises 0.5-3.75% (w/w) THC and 14-25% CBD.

65. The method according to claim 60, wherein the at least one cannabis plant comprised in the extract is the cannabis strain designated herein as “Avidekel”.

66. A method for treating, preventing, ameliorating or delaying the onset of multiple sclerosis or reducing the frequency of relapses in the relapsing-remitting multiple sclerosis (RRMS) in a subject suffering therefrom, or in a subject who is predicted to suffer from said disease, or who has been diagnosed to potentially develop the disease, said method comprising administrating to said subject a therapeutically effective amount of a composition comprising an extract of the cannabis plant designated herein as “Avidekel”.

67. A method for reducing, inhibiting, attenuating or eliminating at least one side effect associated with injecting glatiramer acetate (GA) to a subject, said method comprising administrating to the subject a therapeutically effective amount of a composition comprising an extract of the cannabis plant designated herein as “Avidekel”.

68. The method according to claim 53, wherein the subject is injected with GA for treating, preventing, ameliorating or delaying the onset of multiple sclerosis (MS).

69. The method according to claim 53, wherein said effect of the injection site reaction is selected from a scar, an edema, pain, redness, soreness, itching, swelling, and a hard lump.

70. The method according to claim 53, wherein said administering of the composition of the cannabis plant extract or the composition per se involve topical or transdermal administering of the composition at the GA injection site.

71. The method according to claim 53, wherein the composition of the cannabis plant extract comprises 0.5-3.75% (w/w) THC and 14-25% CBD.
